(19) United States
(12) Patent Application Publication
(10) Pub. No.: US 2024/0374837 A1
(43) Pub. Date: Nov. 14, 2024

(54) Electronic Component for a Drug Delivery Device

(71) Applicant: Sanofi, PARIS (FR)

(72) Inventors: Paul Richard Draper, Warwick (GB); Anthony Paul Morris, Warwick (GB); Samuel Keir Steel, Warwick (GB)

(21) Appl. No.: 18/692,699

(22) PCT Filed: Sep. 22, 2022
(86) PCT No.: PCT/EP2022/076293

§ 371 Date: Mar. 15, 2024

(30) Foreign Application Priority Data
Sep. 24, 2021 (EP) 21315174.9

(51) Int. Cl. A61M 5/315

(52) U.S. Cl. CPC A61M 5/31568; A61M 2205/3306

(57) ABSTRACT

In at least one embodiment, the electronic component for a drug delivery device comprises a carrier having a mounting section and a sensor section connected to the mounting section via a connection region. A sensor is arranged on the sensor section and an electric element is arranged on the mounting section and electrically connected to the sensor. The sensor section is arranged movable with respect to the mounting section between a first position and a sensing position. In the sensing position, the sensor is axially offset compared to the first position.

### CROSS-REFERENCE TO RELATED APPLICATIONS

The present application is the national stage entry of International Patent Application No. PCT/EP2022/076293, filed on Sep. 22, 2022, and claims priority to Application No. EP 21315174.9, filed on Sep. 24, 2021, the disclosures of which are incorporated herein by reference.

### TECHNICAL FIELD

An electronic component for a drug delivery device, a mounting member for an electronic component, an arrangement for a drug delivery device, a drug delivery device and a method for assembling an arrangement for a drug delivery device are provided.

### BACKGROUND

Administering an injection is a process which presents a number of risks and challenges for users and healthcare professionals, both mental and physical. A drug delivery device may aim to make self-injection easier for patients. Drug delivery devices using electronics are becoming increasingly popular in the pharmaceutical industry as well as for users or patients. Electronically measuring delivered doses may make usage of drug delivery devices more comfortable.

### SUMMARY

One object to be achieved is to provide an improved electronic component for a drug delivery device. Preferably, the electronic component allows to reliably and precisely place a sensor in a drug delivery device. Further objects to be achieved are to provide an improved mounting member for an electronic component, an improved arrangement for a drug delivery device, an improved drug delivery device and an improved method for assembling an arrangement for a drug delivery device.

These objects are achieved, inter alia, by the subjects of the independent claims. Advantageous embodiments and further developments are subject of the dependent claims and can also be extracted from the following description and the figures.

Firstly, the electronic component for a drug delivery device is specified.

According to at least one embodiment, the electronic component comprises a carrier. The carrier has a mounting section and a sensor section. The sensor section is connected to the mounting section via a connection region.

The connection region is, in particular, part of the carrier. The connection region may directly adjoin the sensor section and/or the mounting section. For example, in the connection region, the mounting section directly adjoins the sensor section. The connection region may be a contiguous, preferably a simply connected, region or section of the carrier. For example, the sensor section is connected to the mounting section only via or in the connection region, respectively.

The carrier may be a connection carrier comprising one or more electrical conductors. The carrier may comprise an electrically insulating base body, e.g. formed of a plastic. The mounting section and the sensor section may each be partially formed by the base body. The base body may be formed in one piece or integrally formed, respectively. The base body may extend contiguously from the mounting section, over the connection region and to the sensor section. One or more conductor tracks may be arranged on the base body and/or may be integrated in the base body. At least one of the conductor tracks may extend from the mounting section via the connection region to the sensor section. The carrier may be a printed circuit board, PCB for short.

Here and in the following, if the expressions “connected” or “connects” or similar expression are used without prefixed specification like “mechanical” or “electrical”, the expressions particularly relate to a mechanical connection.

According to at least one embodiment, a sensor is arranged on the sensor section. For example, the sensor is electrically connected to the carrier at the sensor section. The sensor may be electrically connected to one or more conductor tracks of the carrier. The sensor may be fixed to the carrier in the sensor section. The sensor may be soldered or glued to the sensor section. The sensor may be a non-contact or contactless sensor. That is to say, the sensor may be configured to perform a sensing operation without physical contact to the object at which the sensing operation or measurement is performed being required.

According to at least one embodiment, an electric element is arranged on the mounting section. The electric element may be electrically connected to the sensor. The electric element may be electrically connected to the carrier at the mounting section. For example, the electric element is electrically connected to one or more conductor tracks of the carrier. The electric element may be electrically connected to the sensor via one or more of these conductor tracks. The electric element may be fixed to the carrier in the mounting section. The electric element may, in particular, be soldered or glued to the mounting section. The carrier may, in particular, mechanically carry the sensor and the electric element and/or may electrically connect the electric element and the sensor, e.g. via the conductor tracks.

The electric element may be, e.g., an electric control unit or a processor or an element of a wireless communication module, e.g. of a Bluetooth module. Particularly, the electric element may be configured to exchange electric signals with the sensor. For example, the electric element is configured to operate and/or activate the sensor and/or to receive measurement signals from the sensor and/or to electronically process the measurement signals.

According to at least one embodiment, the sensor section is arranged movable with respect to the mounting section. Particularly, the sensor section may be movable with respect to the mounting section between a first position and a sensing position. The first position may be a position in which the electronic component is delivered and/or a position prior to assembling the electronic component to an arrangement for a drug delivery device. The sensing position may be a position for operating the sensor and/or a position in which the electronic component is assembled to an arrangement for a drug delivery device.

Movable means, preferably, that the sensor section is arranged pivotable and/or bendable and/or foldable with respect to the mounting section. For example, at least in the connection region, the carrier is formed flexible or bendable in order to allow a relative movement between the mounting section and the sensor section. The connection region may be formed by a hinge, e.g. a film hinge. For example, the carrier is a so-called flex-board. The movement from the first position into the sensing position and/or from the sensing position into the first position may be reversible.

When moving the sensor section relative to the mounting section from the first position into the sensing position, the sensor section may be pivoted relative to the mounting section by at least 45° or at least 80°, e.g. 90°. Additionally or alternatively, the sensor section may be pivoted by at most 135° or at most 100° between the two positions.

The carrier may be thin. For example, a thickness of the carrier, measured between a front side and a back side of the carrier, is smaller, e.g. at least 5 times or at least 10 times smaller, than the expansions of the front side or of the back side, respectively. In the first position, a main extension plane of the mounting section may be parallel or almost parallel to a main extension plane of the sensor section. For example, in the first position, an angle between the main extension planes of the sensor section and the mounting section is at most 10° or at most 5°. In the sensing position, the main extension planes of the sensor section and the mounting section may be oblique to each other, e.g. perpendicular to each other. For example, in the sensing position, an angle between the main extension planes of the sensor section and the mounting section is at least 45° or at least 80°.

The terms “main extension plane” and “main extension direction/axis” are known to the skilled person. Particularly, a main extension plane of an element may be a plane fitted through that element, e.g. via chi\hat{ }2 optimization. Accordingly, a main extension direction may be the direction of a line, e.g. a straight line, fitted through the element. Such a straight line may define the main extension axis.

The electric element and the sensor may be arranged on different sides of the carrier. For example, the sensor is arranged on the back side of the carrier and the electric element is arranged on the front side of the carrier.

According to at least one embodiment, in the sensing position, the sensor is axially offset compared to the first position. “Axially offset” means offset in an axial direction. This means that movement of the sensor section from the first position into the sensing position comprises a movement of the sensor in axial direction. Particularly, in the sensing position, the sensor is further axially offset with respect the mounting section and/or the electric element than in the first position.

The axial direction is herein defined as a direction parallel to or along a longitudinal axis. The longitudinal axis may run perpendicularly to the main extension plane of the mounting section. Particularly, the longitudinal axis intersects with the mounting section and/or runs through a center thereof, e.g. a geometric center and/or a center of mass thereof. The longitudinal axis may intersect with and/or may run perpendicularly to the front side and/or the back side of the carrier in the mounting section.

For example, when moving from the first position into the sensing position, the sensor is axially displaced by at least 0.5 cm or at least 1 cm. Additionally or alternatively, the sensor is axially displaced by most 5 cm or at most 3 cm.

Here and in the following, the longitudinal axis is particularly used for defining a coordinate system and/or for defining directions in order to describe relative positions and relative movements between elements or members or features. As already mentioned, directions parallel to the longitudinal axis are herein defined as axial directions. A direction perpendicular to the longitudinal axis and/or intersecting with the longitudinal axis is herein called radial direction. An inward radial direction is a radial direction pointing towards the longitudinal axis. An outward radial direction is a radial direction pointing away from the longitudinal axis.

The terms “angular direction”, “azimuthal direction” and “rotational direction” are herein used as synonyms. Such a direction is a direction perpendicular to the longitudinal axis and perpendicular to the radial direction. This direction is, in particular, a direction of movement on a circular track around the longitudinal axis.

In at least one embodiment, the electronic component for a drug delivery device comprises a carrier having a mounting section and a sensor section connected to the mounting section via a connection region. A sensor is arranged on the sensor section and an electric element is arranged on the mounting section and electrically connected to the sensor. The sensor section is arranged movable with respect to the mounting section between a first position and a sensing position. In the sensing position, the sensor is axially offset compared to the first position.

The electronic component described herein can, inter alia, be easily and reliably mounted on a mounting member. For example, the electronic component with the sensor section in the first position is first placed on the mounting member and then the sensor section can be moved into its sensing position. The sensor can thereby be brought into a nominal position and can be coupled to the mounting member in order to be held in the nominal position.

According to at least one embodiment, a distance between the connection region and the sensor is constant or substantially constant during movement of the sensor section from the first position to the sensing position. For example, during this movement, the distance changes by at most 5% or at most 1%.

According to at least one embodiment, the carrier is more rigid in the mounting section than in the connection region and/or than in the sensor section. For example, in the mounting section, the carrier is thicker, e.g. at least two times thicker, than in the connection region and/or than in the sensor section.

According to at least one embodiment, besides the electric element and the sensor, one or more further electric elements may be arranged on the carrier. The electric or electronic component may comprise one or more or all of the following further electric elements: a clock, a memory, a capacitor, an inductor, a processor, a control unit, a switch, a part of a Bluetooth module or more generally of a wireless communication module, a further sensor, e.g. a pressure sensor or a touch sensor or a capacitive sensor.

One or more or all of the further electric elements may be arranged on the same side of the carrier as the electric element, e.g. on the front side of the carrier. Additionally or alternatively, one or more or all further electric elements may be arranged on the opposite side of the electric element (particularly on the same side as the sensor), e.g. on the back side. One or more or all further electric elements may be arranged in the mounting section. For example, a further sensor, e.g. a pressure sensor or a touch sensor or a capacitive sensor, is arranged in the mounting section.

According to at least one embodiment, the electric or electronic component comprises an antenna. The antenna may be configured for a wireless communication between the electronic component and a further device, e.g. a smart phone or a computer. For example, data received with help of the sensor may be sent to the further device via the antenna. The antenna may be electrically connected to the electric element and/or the sensor.

The antenna may comprise an antenna section formed by the carrier. The antenna section may be connected to and/or may adjoin the mounting section and/or may extend away from the mounting section. In particular, the antenna section is elongated, i.e. the length of the antenna section is greater than the width and/or the thickness of the antenna section. For example, the length is at least 5 times or at least 10 times the width. The antenna section may be formed flexible. Particularly, the antenna section may be configured to be coiled, e.g. around the longitudinal axis, when mounting the electronic component in the drug delivery device.

According to at least one embodiment, the sensor is configured to detect a relative movement between the sensor and a further member, also referred to as movable member in the following. Particularly, the sensor is configured to detect a relative rotational movement between the sensor and the further member. For example, the sensor is configured to detect a rotational movement of the further member when the sensor axially overlaps or is axially aligned with the further member.

According to at least one embodiment, the sensor is arranged to measure or detect a sensing region, respectively. Preferably, in the sensing position, the sensing region is located axially below the mounting section. Particularly, the sensing region radially overlaps with the mounting section. For example, when viewing along the longitudinal axis, particularly in distal direction, the sensing region is covered by the mounting section. The sensor may be configured to detect physical, electro-magnetic and/or chemical features or changes happening in the sensing region.

For example, the sensor comprises a sensor surface configured to receive signals from an object to be examined and/or to abut an object to be examined. In the sensing position, the sensor surface is preferably facing towards the sensing region located axially below the mounting section and/or towards the object to be examined and axially located below the mounting section. Particularly, in the sensing position, the sensor surface may face the longitudinal axis. In the sensing position, a normal of the sensor surface may run parallel or under an acute angle, e.g., of at most 10°, to the main extension plane of the mounting section. Particularly, in the sensing position, the sensor surface may face in radial inward direction.

According to at least one embodiment, the sensor is an optical sensor. As an optical sensor, the sensor preferably comprises a light-emitting element, e.g. an LED. The light-emitting element may emit radiation, e.g. infrared light, and a reflected portion of this radiation is then detected by the sensor, e.g. a radiation sensitive element thereof, such as a detector chip.

Additionally or alternatively, the sensor may be a radiation sensor, e.g. a light sensor or an infrared sensor, or an accelerometer, or a sound sensor or a pressure sensor or a temperature sensor or a proximity sensor or an ultrasonic sensor or a color sensor or a humidity sensor or a tilt sensor or a flow sensor or a magnetic sensor, e.g. a Hall effect sensor, or a lidar or an electrical current sensor or an optical sensor or a force/torque sensor or a strain gauge sensor or a mechanical switch. However, preferably, the sensor is not a mechanical switch. The sensor may be a digital sensor or an analogue sensor.

According to at least one embodiment, in the first position and/or in the sensing position, the sensor is angularly offset with respect to the connection region, i.e. is offset in angular direction. Particularly, the sensor does not overlap with the connection region in angular direction. For example, the sensor is angularly offset with respect to the connection region by at least 5° or at least 10° or at least 20°. Additionally or alternatively, the sensor may be angularly offset with respect to the connection region by at most 30° or at most 20°. Particularly, the shortest path from the region of carrier on which the sensor is arranged to the mounting section and/or to the connection region is a path that does not entirely run through the carrier, e.g. passes a gap between the mounting section and the sensor section.

According to at least one embodiment, moving the sensor section from the first position to the sensing position comprises a movement in axial and/or radial direction, e.g. in radial inward direction. For example, movement of the sensor section from the first position into the sensing position is predominantly in radial direction and axial direction. During this movement, a displacement in axial direction and/or in radial direction may be larger than a displacement in angular direction, e.g. at least 5 or at least 10 times as large. For example, during the movement, the sensor is not moved in angular direction. This means that the angular position of the sensor and/or the sensor section may be the same in the first position and in the sensing position, e.g. stays the same during movement from the first position into the sensing position.

According to at least one embodiment, in the sensing position, the sensor is axially offset with respect to the connection region. For example, the sensor is axially offset with respect to the connection region by at least 0.5 cm or at least 1 cm. Additionally or alternatively, in the sensing position, the sensor may be axially offset with respect to the connection region by at most 5 cm or at most 3 cm. In the first position, the sensor may be radially offset with respect to the connection region, e.g. by the same distances.

In the first position, the sensor may axially overlap or may be axially aligned with the connection region. Additionally or alternatively, in the first position, the sensor may be less axially offset with respect to the connection region than in the sensing position. For example, in the first position, an axial offset with respect to the sensor compared to the connection region is at most 0.5 cm. In the sensing position, the sensor may be radially aligned or may radially overlap with the connection region.

Here and in the following, when talking about relative positions between two elements, this may particularly relate to the relative positions between the geometric centers or the center of masses of the elements. In case of the sensor, its position may also be defined by an optical center of the sensor.

According to at least one embodiment, the sensor section comprises a first subsection and a second subsection.

According to at least one embodiment, the first subsection connects the second subsection with the connection region. For example, the first subsection is connected to the second subsection via a further connection region. The first subsection may directly adjoin the second subsection and/or the connection region. Alternatively, a further subsection, e.g. a curved subsection, may be formed between the first subsection and the second subsection. The subsections may be arranged one behind the other along the length of the sensor section and/or may each extend over the entire width of the sensor section.

The first subsection and/or the second subsection may be formed more rigid than the further connection region and/or than the connection region, e.g. may be formed thicker. The further connection region may be formed flexible or bendable so that the second subsection may be movable with respect to the first subsection, e.g. pivotable and/or bendable with respect to the first subsection.

According to at least one embodiment, in the first position and/or in the sensing position, the second subsection is arranged angularly offset with respect to the connection region and/or the first subsection. For example, in the first position, a gap is formed between the second subsection and the mounting section so that there is no straight path from the second subsection to the mounting section completely running through the carrier. In the sensing position, the gap may be arranged axially between the second subsection in the mounting section.

According to at least one embodiment, in the first position, the second subsection may be axially aligned or may axially overlap with the first subsection and/or the connection region. In the first position, the second subsection is, in particular, radially offset with respect to the first subsection and/or the connection region.

According to at least one embodiment, in the sensing position, the second subsection is axially offset with respect to the first subsection and/or the connection region. In the sensing position, the second subsection may be radially aligned or may radially overlap with the first subsection and/or the connection region.

According to at least one embodiment, in the first position and/or in the sensing position, the sensor angularly overlaps with the second subsection. The sensor may be arranged on the second subsection. A conductor track from the sensor to the electric element may extend from the sensor via the second subsection, if applicable via the further connection region and/or the curved subsection, via the first subsection, via the connection region to the mounting section.

According to at least one embodiment, in the first position and/or in the sensing position, the second subsection is more orientated in angular direction than the first subsection. Particularly, a main extension axis of the second subsection may be more orientated in angular direction than a main extension axis of the first subsection.

For example, in the first position and/or in sensing position, an angle between the main extension axis of the second subsection and the angular direction is at most 45° or at most 30° or at most 20°. In the first position and/or sensing position, an angle between the main extension axis of the first subsection and the angular direction may be at least 45° or at least 60° or at least 70°. For example, the angle of the main extension axis of the second subsection to the angular direction is at most 50% or at most 30% of the angle between the main extension axis of the first subsection and the angular direction.

According to at least one embodiment, in the first position, the first subsection is more orientated in radial direction than the second subsection. In the sensing position, the first subsection may be more orientated in axial direction than the second subsection. The values for the angles mentioned in the last paragraph may accordingly apply for the orientation concerning the radial direction and/or the axial direction.

Additionally or alternatively, in the first position and/or in the sensing position, the second subsection may be more orientated along a rotation axis than the first subsection. The rotation axis is the axis around which the sensor section is folded/bended/pivoted relative to the mounting section when being moved from the first position into the sensing position. For example, in the first position and/or the sensing position, the second subsection may run parallel to the rotation axis and/or the first subsection may run perpendicularly to the rotation axis.

According to at least one embodiment, the sensor section is an arm of the carrier. The arm may have a free end. Furthermore, the arm may extend between the connection region and the free end.

Particularly, the arm may be elongated. Thus, the length of the arm may be larger than the width and/or the thickness of the arm. For example, the length of the arm is at least twice or at least 5 times the width and/or the thickness of the arm. The free end of the arm may be movable with respect to the mounting section. The free end of the arm may be a longitudinal end of the arm. The further longitudinal end of the arm may adjoin the connection region.

According to at least one embodiment, the orientation of the arm changes when going from the connection region to the free end. This may be valid for the first position and/or the sensing position. For example, in the first position, when starting from the connection region, the arm first extends away from the mounting section, e.g. mainly or only in outward radial direction, then makes a curve and from thereon till the free end extends less strongly away from the mounting section, e.g. mainly or only in angular direction. Accordingly, in the sensing position, when starting from the connection region, the arm first extends away from the mounting section, e.g. mainly or only in axial direction, particularly in distal direction, then makes a curve and from thereon till the free end extends less strongly away from the mounting section, e.g. mainly or only in angular direction.

According to at least one embodiment, in the first position and/or in the sensing position, the arm is more orientated in angular direction in a region closer to the free end than in a region closer to the connection region. For example, in the region from the free end up to a curve in the arm, the arm is more orientated in angular direction than in the region from the curve of to the connection region.

According to at least one embodiment, the arm is dog-leg shaped.

According to at least one embodiment, a slit is formed in the sensor section. A length of the slit may be larger than a width of the slit, e.g. at least 5 times or at least 10 times the width. The slit may extend completely through the sensor section, i.e. from the front side up to the back side and/or over the entire thickness of the sensor section.

According to at least one embodiment, the slit extends along the sensor section, particularly along the arm. For example, the slit follows the shape of the sensor section. The slit may change its orientation along its length. Particularly, the slit comprises a first region and a second region, wherein the first region is closer to the connection region. The second region may be closer to the free end of the arm. When the sensor section is in the first position and/or in the sensing position, the slit in the second region may be more orientated in angular direction than in the first region. Particularly, the slit may be formed contiguously. The slit may extend over a large part of the lengths of the sensor section, e.g. over at least 50% or at least 75% of the length of the sensor section.

According to at least one embodiment, the slit comprises a hole at one longitudinal end or at both longitudinal ends. The diameter of the hole(s) may be larger than the width of the slit in a center region. The hole(s) may extend completely through the sensor section.

A slit formed in the sensor section increases the movability of the sensor section, particularly a movability in axial direction when the sensor section is in its sensing position. This may simplify mounting the electric or electronic component on the mounting member and bringing the sensor in its nominal position with respect to the mounting member.

According to at least one embodiment, in the first position and/or the sensing position, the sensor is aligned with the slit or overlaps with the slit in angular direction, respectively.

According to at least one embodiment, in the sensing position, the sensor is axially offset with respect to the slit. For example, the sensor is arranged axially further away from the mounting section than the slit when the sensor section is in the sensing position. Particularly, in the sensing position, the sensor is more axially offset with respect the slit than in the first position.

According to at least one embodiment, a conductor track of the carrier, e.g. a conductor track electrically connecting the sensor with the electric element, extends next to the slit along the slit. E.g., the conductor track extends parallel to the slit.

According to at least one embodiment, the electronic component comprises at least one coupling feature. The coupling feature(s) may be configured to interact with one or more coupling features of the mounting member in order to hold the sensor in its nominal position relative to the mounting member. The coupling feature(s) may be formed on or by one or more coupling regions of the sensor section. For example, in the first position and/or the sensing position, the coupling feature(s) is/are located angularly offset with respect to the sensor. The sensor may be arranged between the coupling features in angular direction. For example, one coupling feature is formed at or by the free end of the arm.

The coupling feature(s) may be region(s) of the sensor section configured to be inserted into one or more pockets of the coupling member. In these regions, the carrier may be thinner than e.g. in the region in which the sensor is arranged. The coupling feature(s) may be arranged in the second subsection of the sensor section.

According to at least one embodiment, the sensor is arranged on a tab of the sensor section. The tab may be angularly offset with respect to the connection region and/or the first subsection. In the sensing position, the tab may extend from the rest of the sensor section in an axial direction away from the mounting section. For example, in the sensing position, the tab is located axially further away from the mounting section than the rest of the sensor section, e.g. than the coupling feature(s) and/or than the curved subsection and/or than the first subsection. The tab may be part of the second subsection.

According to at least one embodiment, the carrier comprises a second sensor section. All features disclosed in connection with the sensor section are also disclosed for the second sensor section. Particularly, a second sensor may be arranged on the second sensor section and the second sensor section may be connected to the mounting section via a connection region. The second sensor may be of the same type as the sensor of the sensor section. All features disclosed for the sensor and for the sensor section are also disclosed for the second sensor and for the second sensor section.

The second sensor may also be electrically connected to the electric element on the mounting section. The second sensor section may also be movable, particularly pivotable or bendable, between a first position and a sensing position so that, in the sensing position, the second sensor is axially offset compared to the first position. The second sensor may be configured to detect a relative movement between the second sensor and the further member. For example, the measurements from the sensor and the second sensor may be combined in order to increase the resolution of the measurement of the movement and/or to determine the direction of movement of the further member relative to the sensor and the second sensor, e.g. via a Gray-Code output generated by the sensors (i.e. sensor and second sensor) in combination in response to movement of the further member.

According to at least one embodiment, the second sensor section is angularly offset with respect to the sensor section. This may be valid when both sensor sections are in their respective first position and/or in their respective sensing position. For example, when both sensor sections are in their respective sensing position, the two sensors may be angularly offset.

The angular distance between the two sensors, when the respective sensor sections are in the sensing positions, may be predetermined depending on sensing features of the further member. For example, the two sensors are optical sensors.

According to at least one embodiment, the further member comprises an encoder structure. The encoder structure may be moved, e.g. rotate, relative to the sensor(s), e.g. during a dose delivery operation performed by the drug delivery device. The movement of the encoder structure relative to the sensor(s) may be detected and/or measured via the sensor(s), particularly such that the relative movement between the sensor(s) and the further member may be quantified, e.g. the rotation angle can be determined. The measurements may be used to calculate the dose delivered in the dose delivery operation. The encoder structure may be provided on or by on an outer surface of the further member, e.g. circumferentially around the further member. The encoder structure may comprise encoder regions exhibiting different reflectivities for light or infrared radiation which are alternatingly disposed in the circumferential direction. The encoder structure may comprise alternating dark and bright regions, for example. An angular width of each of the encoder regions may be W, e.g. W=30°. Preferably, W is chosen such that W*m=360°, with m being an integer. The angular spacing between the two sensors may be W*n+W/2, wherein n is an integer. The angular spacing between the two sensors particularly refers to the angular spacing between the optical centers of the two sensors. Therefore, during rotation of the further member relative to the sensors, the sensors may be out of phase relative to the encoder regions.

According to at least one embodiment, the sensors and the encoder structure are adjusted such that the sensors and the further member provide a system suitable to generate a multi-bit Gray-Code output, e.g. a 2 bit Gray-Code, during the movement of the further member relative to the sensors. Thus, unique relative positions, e.g. four in case of a 2-bit Gray-Code, between the sensors and the further member may be determined via the combined output signals of the sensors.

According to at least one embodiment, in the first position and/or in the sensing position, the connection regions of the sensor section and the second sensor section are arranged between the sensors in angular direction. The sensor section and the second sensor section may be oppositely orientated in angular direction. For example, in the first position and/or in the sensing position, the sensor section and the second sensor section are arranged mirror symmetrically with respect to a plane extending parallel to the axial direction and the radial direction.

Next, the mounting member for an electric or electronic component is specified. Particularly, the mounting member may be configured for mounting or holding, respectively, the electric or electronic component specified herein. The mounting member may be a chassis lower. For example, the mounting member is formed of plastic and/or in one piece.

According to at least one embodiment, the mounting member comprises a top side. The top side is, in particular, configured such that the mounting section can be placed on it. For example, the area and/or the shape of the top side is adapted to the area and/or shape of the mounting section.

For example, the top side has the shape of a circle. The top side is, in particular, configured to hold or carry the mounting section. When the electronic component is mounted on the mounting member, the longitudinal axis may run obliquely, e.g. perpendicularly, through the top side, e.g. a center thereof.

According to at least one embodiment, the mounting member comprises a lateral side. The lateral side may extend obliquely, e.g. perpendicularly, to the top side. The lateral side may extend circumferentially around the longitudinal axis.

The mounting member may be elongated and/or tubular-shaped. For example, a main extension direction of the mounting member runs along the longitudinal axis.

According to at least one embodiment, the lateral side comprises at least one coupling feature for holding the sensor in a nominal position, when the sensor section is in its sensing position.

According to at least one embodiment, at least one coupling feature of the lateral side is configured to prevent an axial and/or rotational displacement of the sensor relative to the nominal position of the sensor. For example, the at least one coupling feature is realized by one or more protrusions protruding in radial outward direction. The protrusions may abut or may be arranged to abut against the sensor in order to block or restrict a movement of the sensor in rotational and/or axial direction.

According to at least one embodiment, at least one coupling feature of the lateral side is configured to prevent a radial displacement of the sensor relative to its nominal position. For example, the at least one coupling feature is a pocket configured to receive a coupling feature of the electronic component, particularly to receive the free end of the arm. The pocket may be configured to receive the coupling feature of the electronic component by sliding in the coupling feature of the electronic component in axial direction, e.g. in an axial direction away from the mounting section. The lateral face may comprise two such coupling features, e.g. each in form of a pocket, for receiving coupling features of the sensor section. In the nominal position, the sensor may be arranged angularly between these two coupling features of the lateral side.

Next, the arrangement for a drug delivery device specified.

According to at least one embodiment, the arrangement comprises an electronic component. The electronic component may be the electronic component specified herein.

According to at least one embodiment, the arrangement comprises a mounting member. The mounting member may be the mounting member specified herein.

All features disclosed in connection with the electronic component and/or the mounting member are also disclosed for the arrangement and vice versa.

According to at least one embodiment, the electronic component is mounted on the mounting member. For example, the mounting member carries the electronic component. The electronic component may be rotationally and/or axially and/or radially fixed to the mounting member.

According to at least one embodiment, the mounting section is placed on the top side. For example, the mounting section covers a large part, e.g. at least 50% or at least 75% of the top side. The mounting section and/or elements arranged on the mounting section, e.g. the electric element, may abut the top side.

According to at least one embodiment, the sensor section is in its sensing position. For example, the sensor section is pivoted or bent or folded over an edge of the mounting member formed between the top side and the lateral side. A bending axis of the carrier about which the sensor section is bent or pivoted or folded relative to the mounting section may run parallel to said edge.

According to at least one embodiment, the sensor is held in its nominal position by the at least one coupling feature of the mounting member.

According to at least one embodiment, the arrangement comprises a movable member which is arranged movable with respect to the sensor. For example, the movable member is coupled to the arrangement. The movable member may be arranged movable with respect to the sensor in rotational and/or axial direction. The movable member may be the above mention further member.

The movable the member may comprise a sensing surface configured to be examined by the sensor. The sensing surface may face in radial outward direction. The sensing surface may comprise or form the encoder structure mentioned above. For example, the sensing surface in combination with the sensor and the second sensor as discussed further above may be suitable to define a Gray-Code. The sensor of the sensor section or its sensor surface, respectively, may face the sensing surface. The movable member may comprise an encoder ring and/or a dial sleeve.

According to at least one embodiment, the sensor is configured to detect a movement of the movable member with respect to the sensor, particularly a rotational movement.

According to at least one embodiment, in a first state of the arrangement, the movable member may be rotationally locked to the sensor. This means. in the first state, the movable member cannot be rotated relative to the sensor. For example, in the first state, the movable member is rotationally locked to the mounting member via a rotation-lock interface. The rotation-lock interface may be a toothed interface. The first state may be a state for dialing a dose.

According to at least one embodiment, in a second state of the arrangement, the movable member is rotatable relative to the sensor. Particularly, the rotation-lock interface between the movable member and the mounting member is released in the second state. The second state may be a state for dispensing the dialed dose.

Next, the drug delivery device is specified. The drug delivery device may be an injection device and/or a pen type device, e.g. a dial extension pen. The drug delivery device may be a variable dose device in which the drug dose to be delivered to a user can be variably set. For example, the drug delivery device is a reusable device.

According to at least one embodiment, the drug delivery device comprises the electronic component specified herein or the arrangement specified herein. Therefore, all features disclosed in connection with the electronic component or the arrangement are also disclosed for the drug delivery device and vice versa.

According to at least one embodiment, the drug delivery device comprises a container holder for holding a drug container. The container holder may be a housing of the drug delivery device or may be connected or connectable to the housing. The container holder may be configured to hold the drug container axially and/or rotationally fixed with respect to the housing of the drug delivery device. Particularly, the container holder may hold the drug container such that the drug container does not move in axial and/or rotational direction during a drug delivery process.

According to at least one embodiment, the drug delivery device comprises a drug container filled with a drug. The drug container may be a syringe with a pre-mounted needle at a distal end. Alternatively, a needle may be attachable to the drug container, e.g. to a distal end thereof.

The drug delivery device may be elongated. A main extension direction of the drug delivery device may coincide with the longitudinal axis. Additionally or alternatively, the drug delivery device may have a rotational symmetry with respect to the longitudinal axis. A direction parallel to the longitudinal axis is herein called an axial direction. By way of example, the drug delivery device is cylindrically-shaped.

Furthermore, the drug delivery device may comprise an end, e.g. a longitudinal end, which may be provided to face or to be pressed against a skin region of a human body. This end is herein called the distal end. A drug or medicament may be supplied via the distal end. The opposing end is herein called the proximal end. The proximal end is, during usage, remote from the skin region. The axial direction pointing from the proximal end to the distal end is herein called distal direction. The axial direction pointing from the distal end to the proximal end is herein called proximal direction. A distal end of a member or element or feature of the drug delivery device, e.g. of the user interface member, is herein understood to be the end of the member/element/feature located most distally. Accordingly, the proximal end of a member or element or feature is herein understood to be the end of the element/member/feature located most proximally.

In other words, distally is used herein to specify directions, ends or surfaces which are arranged or are to be arranged to face or point towards a dispensing end of the drug delivery device or components thereof and/or point away from, are to be arranged to face away from or face away from the proximal end. On the other hand, proximal is herein used to specify directions, ends or surfaces which are arranged or are to be arranged to face away from or point away from the dispensing end and/or from the distal end of the drug delivery device or components thereof. The distal end may be the end closest to the dispensing end and/or furthest away from the proximal end and the proximal end may be the end furthest away from the dispensing end. A proximal surface may face away from the distal end and/or towards the proximal end and a distal surface may face towards the distal end and/or away from the proximal end. The dispensing end may be a needle end where a needle unit is or is to be mounted to the device, for example.

According to at least one embodiment, the drug delivery device comprises a user interface member, e.g. a knob. The user interface member may be configured for dialing a dose and/or injecting a dose. The mounting member and/or electronic component may be rotationally and/or axially fixed to the user interface member. The user interface member may be arranged rotatable and/or axially movable with respect to a housing of the drug delivery device and/or with respect to the drug container (holder).

For example, the drug delivery device may be used as follows. Firstly, the user interface member is rotated relative to the housing and/or the drug container (holder). The user interface member may be rotated on a helical path with respect to the housing and/or the drug container (holder). During this, the arrangement is in the first state, in which the mounting member is rotationally locked with the movable member. Thus, the movable member is rotated together with the user interface member. After having dialed the dose, the user may press on the user interface member in order to move it in distal direction for delivering the dialed dose. The arrangement may now be in the second state or switched into the second state, in which the mounting member is rotational decoupled from the movable member. During movement of the user interface member in distal direction, the user interface member may not rotate and the movable member may rotate. The dialed dose may thereby be ejected, e.g. injected into a patient. The sensor(s) on the sensor section(s) may measure the rotation of the movable member. The measurement signals of the sensor(s) may then be sent to the electric or electronic component, e.g. the processor, which may determine from the measurement signal(s) the delivered dose. This information may then be communicated to a further device, e.g. with help of the wireless communication module.

Next, the method for assembling an arrangement for a drug delivery device is specified. The method may be used to assemble the arrangement specified herein. Therefore, all features disclosed in connection with the arrangement are also disclosed for the method and vice versa.

According to at least one embodiment, the method comprises a step in which an electronic component is provided. In this step, the sensor section is preferably in its first position.

According to at least one embodiment, the method comprises a step in which a mounting member is provided.

According to at least one embodiment, the method comprises a step, in which the electronic component is placed with the mounting section on the top side. Particularly, the mounting section is thereby placed on the top side, e.g. touches the top side.

According to at least one embodiment, the method comprises a step in which the sensor section is moved into its sensing position. In this step, the sensor section may be pivoted or bent or folded over the edge formed between the top side the lateral side.

According to at least one embodiment, when the sensor section has been moved into its sensing position, the sensor section and/or the sensor may be coupled to the lateral side with help of the one or more coupling features of the lateral side.

Hereinafter, the electric or electronic component, the mounting member, the arrangement, the drug delivery device and the method for assembling an arrangement described herein will be explained in more detail with reference to drawings on the basis of exemplary embodiments. Same reference signs indicate same or similar or similar acting elements in the individual figures. However, the size ratios involved are not necessarily to scale, individual elements may rather be illustrated with exaggerated size for better understanding.

### BRIEF DESCRIPTION OF THE FIGURES

FIG. 1 shows an exemplary embodiment of a drug delivery device,

FIGS. 2 and 3 show an exemplary embodiment of the electric component in a front view and in a back view,

FIGS. 4 to 6 show an exemplary embodiment of the electric component in perspective views,

FIGS. 7 and 8 show an exemplary embodiment of the arrangement and of the proximal section of the drug delivery device in cross-sectional views,

FIGS. 9 and 10 show an exemplary embodiment of the arrangement in a side view and a perspective view.

### DETAILED DESCRIPTION

In the following, exemplary embodiments will be described with reference to an insulin injection device. The present disclosure is however not limited to such application and may equally well be deployed with injection devices that are configured to eject other medicaments or with drug delivery devices in general, preferably pen-type devices and/or injection devices.

Certain exemplary embodiments in this document are illustrated with respect to a drug delivery device in form of an injection device where the user interface member is formed as a knob realizing an injection button and a dose setting (dialling) member at the same time, e.g. similar to the device as described in WO2014033195. Thus, the knob may be used for initiating and/or performing a dose delivery operation of the drug delivery device and may also be used for initiating and/or performing a dose setting operation. The devices may be of the dial extension type, i.e. their length increases during dose setting. Other injection devices with the same kinematical behaviour of the dial extension during dose setting and dose expelling operational mode are known as, for example, the Kwikpen® or Savvio® device marketed by Eli Lilly and the FlexPen®, FlexTouch® or Novopen® device marketed by Novo Nordisk. An application of the general principles to these devices therefore appears straightforward and further explanations will be omitted. However, the general principles of the present disclosure are not limited to that kinematical behaviour.

Certain other embodiments may be conceived for application to injection devices where there are separate injection button and grip components/dose setting members e.g. the device as described in WO2004078239. Thus, the present disclosure also relates to systems with two separate user interface members, e.g. one for the dose setting operation and one for the dose delivery operation. In order to switch between a dose setting configuration of the device and a dose delivery configuration, the user interface member for dose delivery may be moved relative to the user interface member for dose setting.

If one user interface member is provided, the user interface member may be moved distally relative to a housing. In the course of the respective movement, a clutch between two members of a dose setting and a drive mechanism of the device changes its state, e.g. from engaged to released or vice versa. When the clutch, e.g. formed by sets of meshing teeth on the two members, is engaged, a rotation-lock interface is established and the two members may be rotationally locked to one another. When the clutch is disengaged or released, the rotation-lock interface is released and one of the members may be permitted to rotate relative to the other one of the two members. One of the members may be a drive member or drive sleeve which engages a piston rod of the dose setting and drive mechanism. The drive sleeve may be designed to rotate relative to the housing during dose setting and may be rotationally locked relative to the housing during dose delivery. The engagement between drive sleeve and piston rod may be a threaded engagement. Thus, as the drive sleeve cannot rotate during dose delivery, axial movement of the drive sleeve relative to the housing will cause the piston rod to rotate. This rotation may be converted into axial displacement of the piston rod during the delivery operation by a threaded coupling between piston rod and housing.

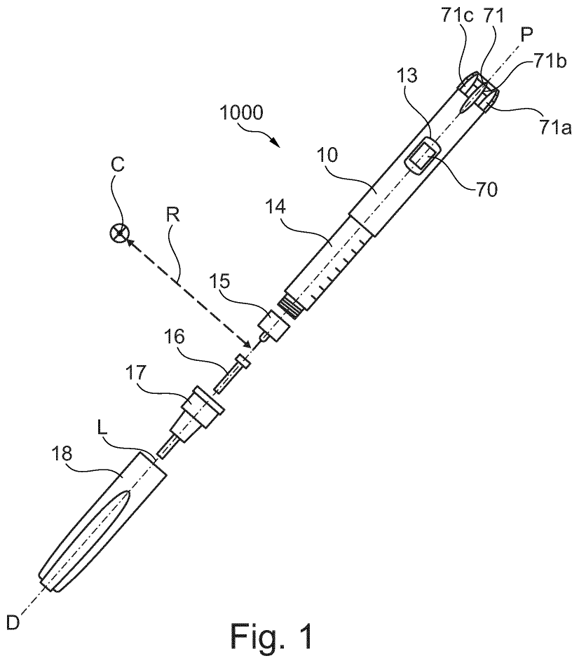

FIG. 1 is an exploded view of an exemplary embodiment of a drug delivery device 1000. In this exemplary embodiment, the drug delivery device 1000 is an injection device, e.g. a pen-type injector.

The injection device 1000 of FIG. 1 is an injection pen that comprises a housing 10 holding a drug container 14, e.g. an insulin container, or a container holder for such a container 14. The container 14 may contain a drug, e.g. insulin. The container 14 may be a cartridge or a receptacle for a cartridge which may contain the cartridge or be configured to receive the cartridge. A needle 15 can be affixed to the container 14 or the receptacle. The container 14 may be a cartridge and the receptacle may be a cartridge holder. The needle 15 is protected by an inner needle cap 16 and either an outer needle cap 17 or another cap 18. An insulin dose to be ejected from the injection device 1000 can be set, programmed, or ‘dialled in’ by turning a user interface member 71 in form of a knob 71, and a currently programmed or set dose is then displayed via dosage window 13, for instance in multiples of units. The units may be determined by the dose setting mechanism which may permit relative rotation of the knob 71 to the housing 10 only in whole-number multiples of one unit setting increment, which may define one dosage increment. This may be achieved by an appropriate ratchet system, for example. The indicia displayed in the window may be provided on a number sleeve or dial sleeve 70. For example, where the injection device 1000 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve 70 that is configured to move when the knob 71 is turned, to provide a visual indication of a currently programmed dose. The knob 71 is rotated on a helical path with respect to the housing 10 when turned during programming.

In this exemplary embodiment, the knob 71 includes one or more features 71a, 71b, 71c in form of formations to facilitate gripping and/or attachment of a data collection device or electronic system.

The injection device 1000 may be configured so that turning the knob 71 causes a mechanical click sound to provide acoustical feedback to a user. In this embodiment, the knob 71 also acts as an injection button. When needle 15 is stuck into a skin portion of a patient, and then the knob 71 is pushed in an axial direction, the insulin dose displayed in display window 13 will be ejected from injection device 1000. When the needle 15 of injection device 1000 remains for a certain time in the skin portion after the knob 71 is pushed home, the dose is injected into the patient's body. Ejection of the insulin dose may also cause a mechanical click sound, which is however different from the sounds produced when rotating the knob 71 during dialing of the dose.

In this exemplary embodiment, during delivery of the insulin dose, the knob 71 is returned to its initial position in an axial movement, without rotation, while the dial sleeve 70 or number sleeve 70 is rotated to return to its initial position, e.g. to display a dose of zero units. As noted already, the disclosure is not restricted to insulin but should encompass all drugs in the drug container 14, especially liquid drugs or drug formulations.

The injection device 1000 may be used for several injection processes until either the insulin container 14 is empty or the expiration date of the medicament in the injection device 1000 (e.g. 28 days after the first use) is reached.

Furthermore, before using injection device 1000 for the first time, it may be necessary to perform a so-called “prime shot” to ensure fluid is flowing correctly from insulin container 14 and needle 15, for instance by selecting two units of insulin and pressing knob 71 while holding injection device 1000 with the needle 15 upwards. For simplicity of presentation, in the following, it will be assumed that the ejected amounts substantially correspond to the injected doses, so that, for instance the amount of medicament ejected from the injection device 1000 is equal to the dose received by the user.

As explained above, the knob 71 also functions as an injection button so that the same component is used for dialling/setting the dose and dispensing/delivering the dose. Again, we note that a configuration with two different user interface members which, preferably only in a limited fashion, are movable relative to one another is also possible. The following discussion will, however, focus on a single user interface member which provides dose setting and dose delivery functionality. In other words, a setting surface of the member which is touched by the user for the dose setting operation and a dose delivery surface which is touched by the user for the dose delivery operation are immovably connected. Alternatively, they may be movable relative to one another, in case different user interface members are used. During the respective operation, the user interface member is preferably moved relative to the body or housing of the device. During dose setting, the user interface member is moved proximally and/or rotates relative to the housing. During dose delivery, the user interface member moves axially, e.g. distally, preferably without rotating relative to the housing or body.

FIG. 1 also indicates the coordinate system used herein for specifying positions of members or elements or features. The distal direction D and proximal direction P run parallel to the longitudinal axis L. The longitudinal axis L is or coincides with a main extension axis of the device 1000. The radial direction R is a direction perpendicular to the longitudinal axis L and intersecting with the longitudinal axis L. The azimuthal direction C, also referred to as angular direction or rotational direction, is a direction perpendicular to the radial direction R and to the longitudinal axis L. The different directions and axes will not be indicated in every of the following figures in order to increase the clarity of the figures.

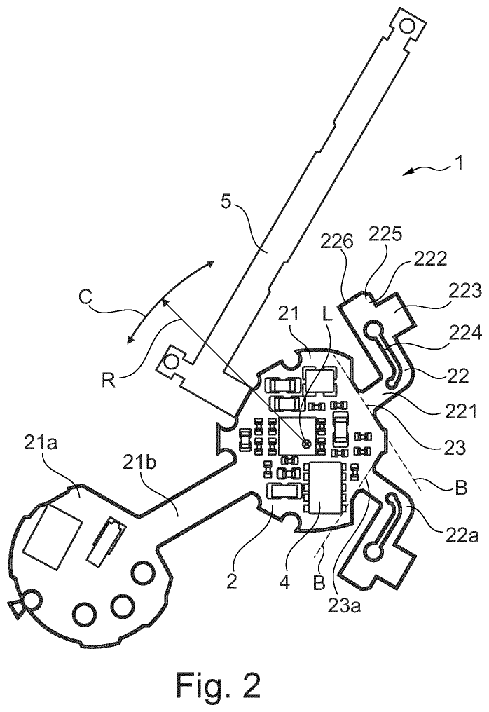

FIG. 2 shows an exemplary embodiment of the electric component 1 in a front view onto a front side of a carrier 2 of the electronic component 1. The carrier 2 is a printed circuit board, PCB for short. Particularly, the carrier 2 is a so-called flex board.

The carrier 2 comprises different sections. A mounting section 21 has an almost circular shape. The longitudinal axis L (indicated by the cross) runs through the mounting section 21 and perpendicularly to the main extension plane of the mounting section 21 and/or perpendicularly to the front side of the carrier 2 in the mounting section 21. An electric element 4, e.g. a control unit or a processor or a real-time clock, is arranged on the front side of the carrier 2 in the mounting section 21. Moreover, one or more further electric elements, like capacitors, inductors and/or light-emitting diodes, are arranged on the mounting section 21, e.g. on the front side and/or on the back side.

Two sensor sections 22, 22a are connected to the mounting section 21 via connection regions 23, 23a. The sensor sections 22, 22a are each movable, particularly bendable, with respect to the mounting section 21 between a first position and a sensing position. FIG. 2 shows the sensor sections 22, 22a in their respective first position. In this first position, the main extension plane of the sensor section 22, 22a extends parallel or almost parallel to the main extension plane of the mounting section 21.

FIG. 2 also indicates the bending axes B around which the sensor sections 22, 22a can be pivoted or bended relative to the mounting section 21. Particularly, the bending axes B run through the respective connection region 23, 23a.

As can be seen in FIG. 2, the sensor sections 22, 22a are elongated arms of the carrier 2, wherein one end of the arm is connected to the mounting section 21 via the connection region 23, 23a and the other end of the arm is a free end 226. The region of the arm adjoining the free end 226 forms a coupling feature 225, which will be explained in more detail below.

In the following, one sensor section 22, also referred to as first sensor section 22, will be described in more detail. However, the features described for the first sensor section 22 may likewise be valid for the second sensor section 22a.

The arm is formed such that their orientation changes when going from the connection region 23 to the free end 226. Particularly, starting from the connection region 23, the arm is firstly formed by a first subsection 221 mainly extending in radial direction R, then the arm 22 is formed by a curved subsection and then a second subsection 222 follows, in which the arm is less orientated in radial direction R but more in angular direction C than in the first subsection 221. The shape of the arm can be described as dog-leg-shaped.

The sensor section 22 also comprises a tab 223, which projects from the rest of the arm in a direction away from the mounting section 21. The tab 223 constitutes a mounting region for the sensor 3 as will be explained below. The tab 223 is angularly offset with respect to the connection region 23 of the sensor section 22.

As can be further seen in FIG. 2, an orientation of a first sensor section 22 in angular direction is opposite to an orientation of the second sensor section 22a in angular direction. Particularly, the sensor section 22, 22a are arranged mirror symmetrically with respect to an axis running in radial direction.

FIG. 2 also shows that slits 224 are formed in the sensor sections 22. The slits 224 are elongated and also change their orientation along their lengths. Particularly, the slits 224 follow the shape of the sensor sections 22, 22a. At their longitudinal ends, the slits 224 are shaped as holes having a larger diameter than in the center of the slits 224.

The electric component 1 also comprises an antenna 5. The antenna 5 comprises an antenna section of the carrier 2 which is elongated and which is connected to the mounting section 21. Starting from the mounting section 21, the antenna section first extends in radial direction and then in a direction perpendicular to the radial direction. The antenna section may be configured to be wrapped around the longitudinal axis L when assembled into a drug delivery device.

The carrier 2 also comprises a further mounting section 21a, which is similarly shaped as the mounting section 21. The further mounting section 21a and the mounting section 21 are connected via an elongated connection section 21b of the carrier 2. The carrier 2 may be flexible or bendable in this connection section 21b so that the further mounting section 21a can be moved into a position where the further mounting section 21a is arranged above the mounting section 21 and axially offset with respect to the mounting section 21. A battery for powering the electric elements on the carrier 2 may then be arranged axially between the mounting section 21 in the further mounting section 21a and may be electrically connected to the mounting section 21 as well as to the further mounting section 21a.

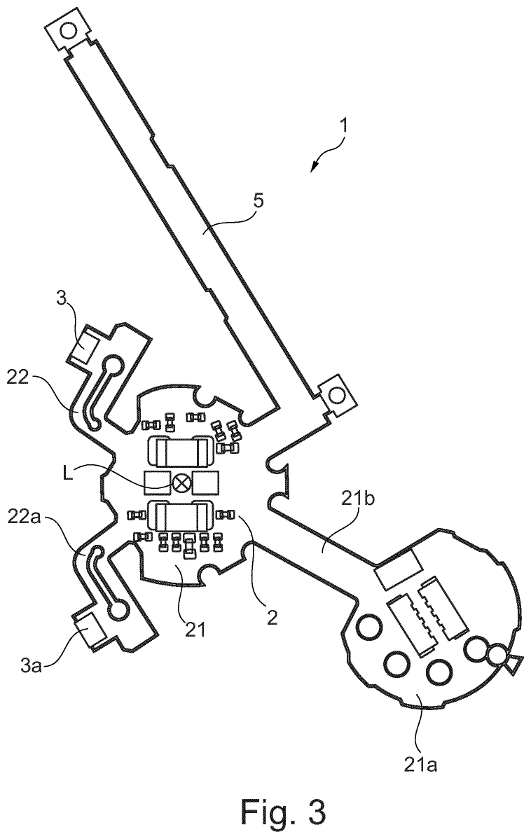

FIG. 3 shows the electric component of FIG. 2, but now turned around so that the back side of the carrier 2 can be seen. Sensors 3, 3a are mounted on the tabs 223 of the sensor sections 22, 22a. The sensors 3, 3a are angularly offset with respect to the connection regions 23, 23a. The sensors 3, 3a may be electrically connected with the electric element 4 on the front side of the carrier 2. For example, conductive tracks run from the sensors 3 along the sensor sections 22, 22a via the connection regions 23, 23a to the electric element 4 and electrically connect the electric element 4 with the sensors 3, 3a. The sensors 3, 3a may be optical sensors, e.g. for detecting light or infrared radiation reflected from a surface. The radiation source generating the light or the radiation, e.g. an LED, may be integrated into the sensor, which, in addition to the radiation source, may comprise a detector chip for receiving reflected radiation.

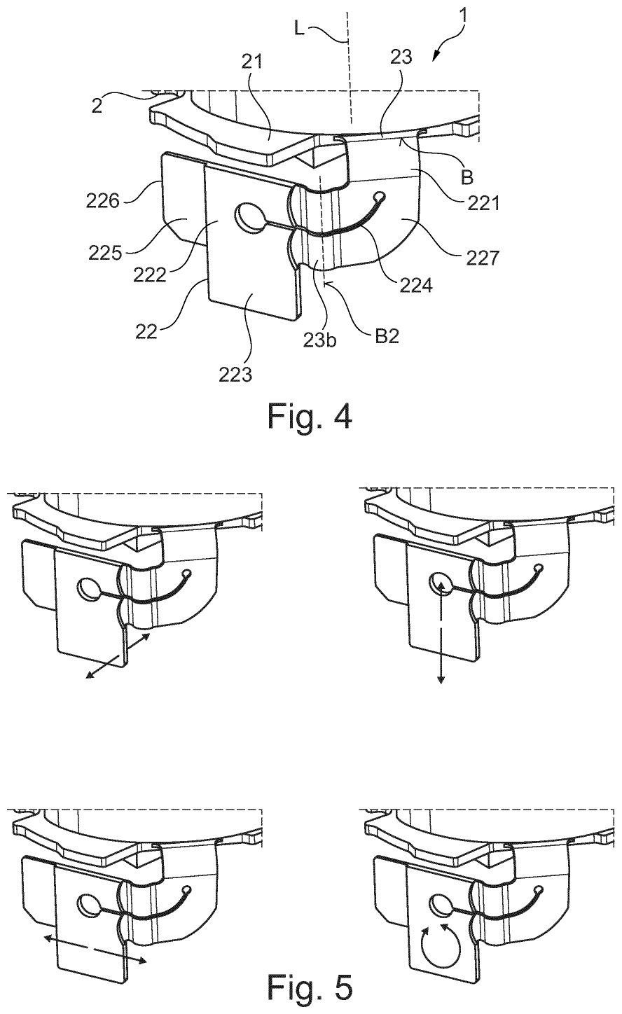

FIG. 4 shows a section of the electric component 1, e.g. of the electric component 1 of FIGS. 2 and 3, in a perspective view. The shown sensor section 22 is now in its sensing position. It can be seen that the carrier 2 is formed less thick in the first subsection 221 adjoining the mounting section 21 or the connection region 23, respectively, than in the mounting section 21. This allows bending the sensor section 22 relative to the mounting section 21 from the first position into its sensing position.

As can be further seen in FIG. 4, the sensor section 22 is formed thicker in the tab 223 than in the region of the second subsection 222 forming the free end 226 and also forming the coupling feature 225.

FIG. 4 also shows that, between the second subsection 222 and the first subsection 221, particularly between the curved subsection 227 and the second subsection 222, a further connection region 23b is formed, which is flexible so that the second subsection 222 can be moved or bent or pivoted with respect to the first subsection 221 around a further bending axis B2. Particularly, this allows to radially move the sensor 3. In the further connection region 23b, the carrier 2 is also formed less thick than in the mounting section 21 or the tab 223, respectively.

FIG. 5 illustrates the movability of the sensor section 22 with respect to the mounting section 21 when the sensor section 22 is in the sensing position. This movability allows to adjust the exact position of the sensor 3. Particularly the special shape of the sensor section 22 with a changing orientation and with the slit 224 allows to adjust the position of the sensor in axial direction, in angular direction, in radial direction and also allows a rotation of the sensor around the radial direction.

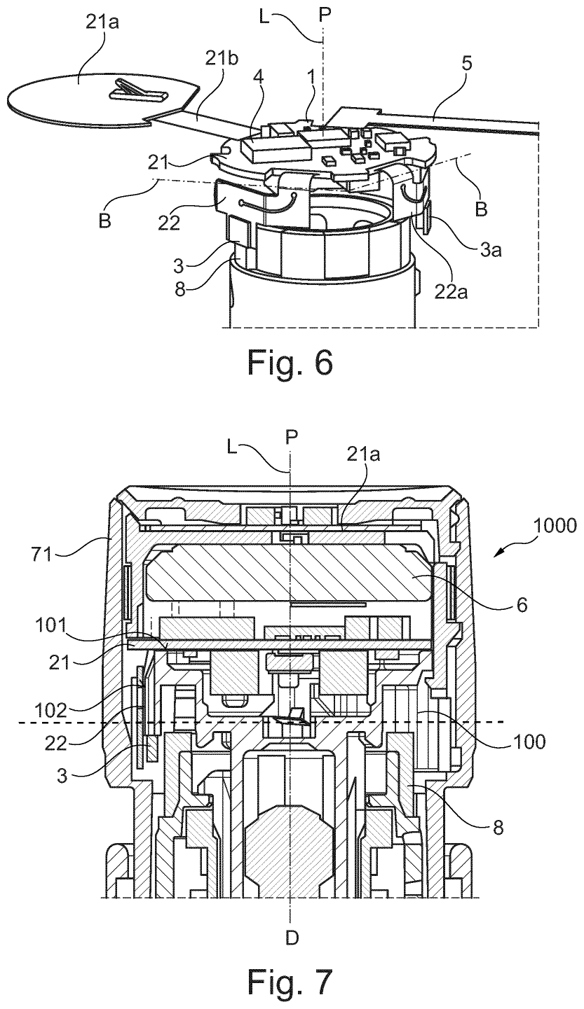

FIG. 6 shows the electric component 1, e.g. the electric component of the previous exemplary embodiments, with the sensor section 22 in the sensing position. In the region of the sensors 3, the carrier 2 is drawn transparent in order to better see the position of the sensors 3, 3a. The sensors 3, 3a are arranged to measure a sensing region which is located axially below the mounting section 21. Sensor surfaces of the sensors 3, 3a preferably face in radial inward direction towards this sensing region and towards the longitudinal axis L.

A movable member 8 is arranged within the sensing region, i.e. axially below the mounting section 21 and axially overlapping or aligned with the sensors 3, 3a. Particularly, the region of the movable member 8 axially overlapping with the sensors 3, 3a may comprise a surface structure and/or alternating reflective and less reflective regions, e.g. alternating dark and bright regions or recessed and non-recessed regions. The sensors 3, 3a may be optical sensors for detecting radiation reflected from the further member and for sending associated measurement signals to the electric component 4 or processor. If the region reflects more radiation to the sensor, e.g. as it is closer to the associated sensor and/or more reflective, the measurement signal is higher than if the region reflects less radiation to the sensor, e.g. as it is further away from the associated sensor and/or less reflective. The movable member 8 is particularly rotatable with respect to the sensors 3. The movable member 8 may be or may comprise a dial sleeve and/or an encoder ring. The movable member may move, e.g. rotate, relative to the sensor(s) during the dose delivery operation e.g. only during the dose delivery operation. During the dose setting operation, the sensor(s) and the movable member may move together, e.g. rotationally and/or axially, relative to a housing of the drug delivery device.

FIG. 7 shows a proximal section of an exemplary embodiment of the drug delivery device 1000. Particularly, the region of a proximal user interface member 71 is illustrated. FIG. 7 is a cross-sectional view, e.g. of the proximal section of the drug delivery device 1000 of FIG. 1. As can be seen, the electric component 1 is mounted on a mounting member 100. The mounting member 100 is a chassis lower comprising a top side 101 and a lateral side 102. The mounting section 21 is mounted on the top side 101 and the sensor section 22 is in its sensing position so that the sensor section 22 axially overlaps with the lateral surface 102.

Furthermore, it can be seen that the further mounting section 21a is arranged axially offset with respect to the mounting section 21 and a battery 6 is arranged between the mounting section 21 and the further mounting section 21a in axial direction.

The drug delivery device 1000 shown in FIG. 7 also comprises a movable member 8, e.g. the movable member 8 of FIG. 6.

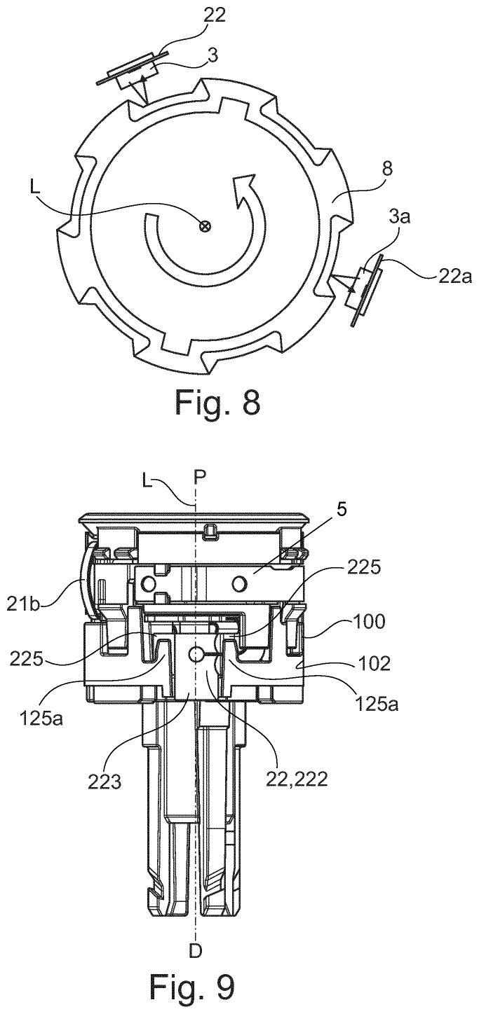

FIG. 8 shows a view on the cross-section plane indicated by the horizontal dashed line of FIG. 7. The sensors 3, 3a are angularly offset to each other. Each sensor 3, 3a is configured to emit infrared radiation which is reflected from the movable member 8 and then detected by the sensors 3, 3a. This allows to detect a rotation of the movable member with respect to the sensors 3, 3a.

In the exemplary embodiment of FIGS. 7 and 8, the movable member 8 comprises twelve black and white regions arranged alternatingly around the movable member 8. Each region is 30° wide which creates twelve transition edges per rotation. The angular spacing between the optical centres of the two optical sensors 3, 3a is 135°. This arrangement places one sensor 3 90° out of phase with the other sensor 3a (30n°+15° for any n achieves this). This facilitates the implementation of a quadrature encoder, meaning the two sensors 3, 3a can be used in combination to determine the direction of rotation. Each edge transition, past one or other sensor, (which happens 24 times in one complete revolution of the movable member) represents the dispense of one unit of a drug. As the sensors are out of phase relative to the regions of the surface structure (with the more reflective or less reflective regions) and the angular width of the regions of the further member is chosen appropriately, the sensors, in combination, generate a Gray-Code output, e.g. a 2-bit Gray-Code output. This allows to distinguish four different relative positions between the sensors and the member 8.

In the case of optical sensors and dependent upon the last dispensed dose, each sensor 3, 3a could be pointing at either a black or white region of the movable member 8. Nominally, the centre of each sensor 3, 3a is positioned a certain angle away from a transition edge between a black and a white region. The sensor response whilst pointing at a white region may be defined as a binary 1, and a binary 0 whilst pointing at a black region. Configurations, whereby the sensors 3, 3a are nominally pointing at one of two states and transitions occur between these states, are applicable to other sensor technologies, as listed above.

In the operation of the device 1000, the movable member 8 may move axially (i.e. proximally or distally) relative to the sensors 3, 3a (or vice versa). The sensors 3, 3a are positioned to view the movable member 8 radially, and in such a way that the sensing distance remains relatively constant irrespective of axial movement. Such an arrangement effectively eliminates the ‘lensing’ effect i.e. the target coming in and out of sensor focus. However, the tolerance stack ups as a result of natural part-to-part variation and variation as a result of assembly processes (for example, solder thickness between the sensor and PCB) mean that the radial and axial movement may be significant in relation to the sensor's optimum working range(s). It is therefore desirable to minimise these tolerances wherever possible. This is achieved, inter alia, with the special design of the electronic component 1 described herein.

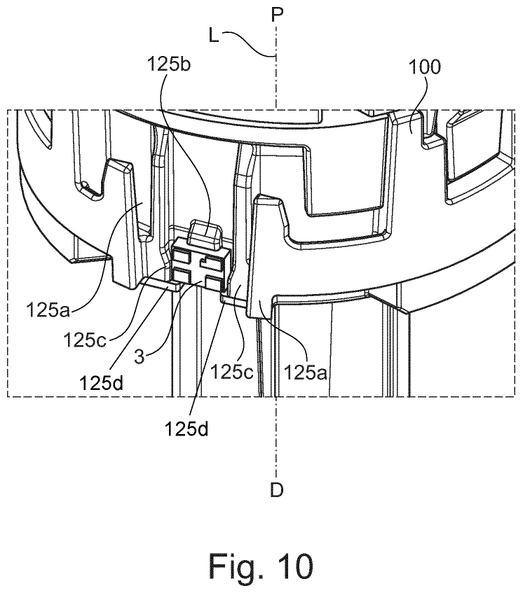

FIG. 9 shows a side view of an exemplary embodiment of the arrangement. FIG. 10 shows a perspective view of the arrangement in which some details are more prominent. In FIG. 10, the carrier 2 is made transparent in order to better see the position of the sensors 3.

The electric component 1 is mounted on the mounting member 100 and the sensor section 22 is in its sensing position. The lateral surface 102 of the mounting member 100 comprises several coupling features 125a, 125b, 125c. Two of these coupling features 125a are pockets in which the coupling features 225 of the sensor section 22 are received. For example, regions of sensor section 22 arranged on both sides of the sensor 3 in angular directions constitute coupling features 225 which have been slid into the pockets 125a in distal direction D. The respective pocket may be open proximally and closed distally. The coupling of the sensor section 22 to the mounting member 100 via the pockets 125a restricts a displacement of the sensor 3 from its nominal position in radial direction and preferably also in distal direction D. Alternatively or additionally, one or more distal stop features 125d may be provided, e.g. radial protrusions, to limit or prevent distal displacement of the sensor 3 relative to the mounting member 100. The respective distal stop feature 125d is expediently positioned so as to angularly and/or radially overlap with the sensor 3.

A further restriction of movement in radial inward direction may be achieved by a surface region of the lateral surface 102 arranged radially inward with respect to the sensor section 22. This surface region radially abuts or is configured to radially abut a region of the sensor section 22 located axially above the sensor 3. Alternatively or additionally, the sensor 3 itself may abut the mounting member 100 and/or its surface 102. This may prevent or assist in preventing radial inward movement of the sensor 3 relative to the mounting member 100. A sensing surface of the sensor 3 may be exposed, particularly radially inwardly not covered by the surface 102 (e.g. because of an aperture in the surface), to permit that the sensor can emit radiation towards and/or receive radiation reflected from the movable member via the sensing surface of the sensor 3.

A further coupling feature of the lateral surface 102 is a protrusion 125b which protrudes in radial outward direction and which is configured to restrict the movement of the sensor 3 in axial direction, particularly in proximal direction P. When in its nominal position, the sensor 3 is located downstream of the protrusion 125b in distal direction D. This protrusion 125b is configured to abut the sensor 3 when the sensor 3 is moved from its nominal position in proximal direction P.

Yet another coupling features 125c are further protrusions protruding in radial outward direction. These protrusions 125c axially overlap with the sensor 3 and are arranged angularly offset with respect to the sensor 3 on both sides of the sensor 3. The protrusions 125c are configured to abut against the sensor 3 when the sensor 3 is moved from its nominal position in angular direction.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The disclosure described herein is not limited by the description in conjunction with the exemplary embodiments. Rather, the disclosure comprises any new feature as well as any combination of features, particularly including any combination of features in the patent claims, even if said feature or said combination per se is not explicitly stated in the patent claims or exemplary embodiments.

1-20. (canceled)

21. An electronic component for a drug delivery device, the electronic component comprising:
a carrier having a mounting section and a sensor section connected to the mounting section via a connection region;
a sensor arranged on the sensor section; and
an electric element arranged on the mounting section and electrically connected to the sensor;
wherein the sensor section is arranged movable with respect to the mounting section between a first position and a sensing position; and
wherein in the sensing position, the sensor is axially offset compared to the first position.

22. The electronic component of claim 21, wherein the electric element is configured to exchange electric signals with the sensor; and
wherein in the sensing position, the sensor is offset in an axial direction compared to the first position, wherein the axial direction is a direction parallel to or along a longitudinal axis perpendicularly to a main extension plane of the mounting section.

23. The electronic component of claim 21, wherein the sensor is configured to detect a relative movement between the sensor and a further member;
wherein the sensor is arranged to measure a sensing region, which, in the sensing position, is located axially below the mounting section; and
wherein the sensor is an optical sensor.

24. The electronic component of claim 21, wherein in the first position and/or in the sensing position, the sensor is angularly offset with respect to the connection region; and
wherein in the sensing position, the sensor is axially offset with respect to the connection region.

25. The electronic component of claim 21, wherein the sensor section comprises a first subsection and a second subsection, the first subsection connecting the second subsection with the connection region;
wherein in the first position and/or in the sensing position, the second subsection is arranged angularly offset with respect to the connection region and/or the first subsection;
wherein in the sensing position, the second subsection is arranged axially offset with respect to the connection region and/or the first subsection; and
wherein in the first position and/or in the sensing position, the sensor angularly overlaps with the second subsection.

26. The electronic component of claim 25, wherein in the first position and/or in the sensing position, the second subsection is more orientated in angular direction than the first subsection; and
wherein in the first position, the first subsection is more orientated in radial direction than the second subsection.

27. The electronic component of claim 21, wherein the sensor section is an arm of the carrier having a free end and extending between the connection region and the free end; and
wherein an orientation of the arm changes when going from the connection region to the free end.

28. The electronic component of claim 27, wherein in the first position and/or in the sensing position, the arm is more orientated in angular direction in a region closer to the free end than in a region closer to the connection region; and
wherein the arm is dog-leg shaped.

29. The electronic component of claim 21, wherein a slit is formed in the sensor section and extends along the sensor section;
wherein in the first position and/or in the sensing position, the sensor is aligned with the slit in angular direction; and
wherein in the sensing position, the sensor is axially offset with respect to the slit.

30. The electronic component of claim 21, wherein the carrier comprises a second sensor section with a second sensor arranged thereon, wherein the second sensor section is connected to the mounting section via a connection region;
wherein the second sensor section is angularly offset with respect to the sensor section; and
wherein in the first position and/or in the sensing position, the connection regions of the sensor section and the second sensor section are arranged between the first sensor and the second sensor in angular direction.

31. The electronic component of claim 21, wherein the carrier is more rigid in the mounting section than in the connection region and/or than in the sensor section.

32. A mounting member for an electronic component comprising a carrier having a mounting section and a sensor section connected to the mounting section and movable relative to the mounting section between a first position and a sensing position, the mounting member comprising:
a top side configured for receiving the mounting section; and
a lateral side extending obliquely to the top side, the lateral side comprising at least one coupling feature for holding a sensor arranged on the sensor section in a nominal position when the sensor section is in the sensing position.

33. The mounting member according to claim 32, wherein the at least one coupling feature of the lateral side is a protrusion protruding in an outward radial direction for preventing an axial and/or rotational displacement of the sensor relative to the nominal position.

34. The mounting member of claim 32, wherein the at least one coupling feature of the lateral side is a pocket for inserting at least one coupling feature of the sensor section to prevent a radial displacement of the sensor relative to the nominal position.

35. A drug delivery device arrangement, the arrangement comprising:
an electronic component comprising a carrier having a mounting section and a sensor section connected to the mounting section and movable relative to the mounting section between a first position and a sensing position, the electronic component comprising a sensor positioned on the sensor section;
a mounting member comprising a top side, a lateral side extending obliquely to the top side, and at least one coupling feature;
wherein the electronic component is mounted on the mounting member such that the mounting section is placed on the top side; and
wherein when the sensor section is in the sensing position, the sensor is held in a nominal position by the at least one coupling feature of the mounting member.

36. The arrangement of claim 35, comprising a movable member moveably arranged with respect to the sensor; and
wherein the sensor is configured to detect a movement of the movable member with respect to the sensor.

37. The arrangement of claim 35, comprising a container holder for holding a drug container filled with a drug.

38. The arrangement of claim 35, comprising a drug container filled with a drug.

39. The arrangement of claim 36, comprising an injection device and/or a pen type injection device.

40. The arrangement of claim 36, comprising a variable dose device in which a drug dose to be delivered to a user is configured to be variably set.
